(19) United States
(12) Patent Application Publication
(10) Pub. No.: US 2017/0252218 A1
(43) Pub. Date: Sep. 7, 2017

(54) Hearing Protection Systems

(71) Applicant: Craig D. Gates, Mead, WA (US)

(72) Inventor: Craig D. Gates, Mead, WA (US)

(21) Appl. No.: 15/058,575

(22) Filed: Mar. 2, 2016

(51) Int. Cl. A61F 11/12; A61F 11/08

(52) U.S. Cl. CPC A61F 11/12; A61F 11/08; A61F 2011/085; A61F 2011/145

(57) ABSTRACT

A hearing protection system includes a trigger mechanism that causes earplugs to displace between an open position and a closed position. The trigger mechanism is responsive to movement of a device and causes the earplugs to displace from the open position to the closed position based at least in part on a position of the device.

### BACKGROUND

Protecting a hearing of a person can be difficult in conditions where hearing protection is desired at a time of an event but not desired until the event. This is particularly true for shooters, where hearing protection is not desired until the firing of a firearm. For example, a hunter may desire hearing protection at the time of firing a firearm but the hearing protection is not desired until the firing of the firearm so that the hunter can hear ambient sound naturally and unchanged for better hunting.

A person may use a variety of hearing protection devices to protect the persons hearing, however each of the hearing protection devices have disadvantages. For example, earplugs can be used, however the earplugs remain stuck in the ears of a user even when the earplugs are not needed and thus prevent the person from hearing ambient sound. For example, a hunter may plug his ears with earplugs to protect his ears at a time of firing a firearm, but the earplugs prevent the hunter from hearing ambient sounds like a location of game, a location of a dog, or verbal communication from another hunter. In another example, noise cancelling earplugs can be used, however the noise cancelling earplugs do not allow the user to hear unchanged ambient sound. Instead, noise cancelling earplugs inject reproduced ambient sound into the ears which is not the same as hearing the ambient sound naturally. For example, the noise cancelling earplugs receive ambient sound and inject digitally reproduced sound into the ears of the user that is distorted and thus not the same as the natural ambient sounds. Moreover, the earplugs are uncomfortable to use because the earplugs prevent heat from dissipating from the ears, causing the ears to perspire and the user to overheat.

Accordingly there remains a need in the art for a hearing protection system, allowing the user to hear ambient sound naturally and more comfortably, until the hearing protection system is needed to protect the ears from a loud noise.

### SUMMARY

Hearing protection systems are configured to protect a hearing of a user from a loud noise. Generally, the hearing protection systems include earplugs that are displaced between an open position and a closed position based on a position of a device. When in the open position, the earplugs are positioned away from the ears of the user to allow the user to hear ambient sound naturally at an unchanged volume, and when in the closed position, the earplugs are positioned proximal to the ears of the user to at least partly block the ambient sound and allow the user to hear the ambient sound at a reduced volume that is less than the unchanged volume. This summary is provided to introduce simplified concepts of hearing protection systems, which are further described below in the Detailed Description. This summary is not intended to identify essential features of the claimed subject matter, nor is it intended for use in determining the scope of the claimed subject matter.

In one example, a hearing protection system includes an earplug displaceable between an open position and a closed position, and a trigger mechanism to cause the earplug to displace between the open position and the closed position based at least in part on a position of a device.

In another example, a hearing protection system includes an earplug attachable proximate to an ear of a user. The hearing protection system also includes a trigger mechanism to cause the earplug to displace between an open position and a closed position based at least in part on a position of a device.

In another example, a hearing protection system includes an earplug displaceable between an open position and a closed position in response to a signal received from a trigger mechanism.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

FIG. 1 illustrates an example hunting environment involving hunters.

FIG. 2 illustrates a perspective view of an example hearing protection system in an open position that allows the hunters shown in FIG. 1 to hear ambient sound at an unchanged volume.

FIG. 3 illustrates a perspective view of the example hearing protection system shown in FIG. 2 in a closed position that allows the hunters shown in FIG. 1 to hear ambient sound at a reduced volume that is less than the unchanged volume.

### DETAILED DESCRIPTION

### Overview

This disclosure is directed to hearing protection systems that displace from an open position to a closed position to protect a person's hearing. For example, the hearing protection systems may include a trigger mechanism, responsive to movement of a device, to cause the hearing protection systems to displace from the open position to the closed position based at least in part on a position of the device. For example, the trigger mechanism may cause the hearing protection systems to displace to the closed position based on a position of a device relative to a portion of a body of a user. Stated otherwise, the trigger mechanism may base the closing criteria on a position of a firearm relative to a shoulder or a hand of a hunter. Moreover, the trigger mechanism may employ a sensor to continually sense the position of the firearm relative to the shoulder or the hand of the hunter. The trigger mechanism may cause the hearing protection systems to displace to the closed position where the hearing protection systems are positioned proximal to the ears of the hunter. In this way, the hearing protection systems at least partly close the ears of the hunter when the firearm is fired to prevent injury to the ears of the hunter. While this application describes various embodiments of hearing protection systems used in the field of hunting, this is by way of example and not limitation. For example, the hearing protection systems may be used in other fields such as military applications, construction applications, industrial applications, heavy equipment operation applications, machinery operation applications, music industry applications, aviation industry applications, etc.

The hearing protection systems may include an earplug displaceable between an open position and a closed position. For example, the earplug may be displaced from the open position, where the earplug is positioned away from an ear of a user to allow the user to hear ambient sound at an unchanged volume, to the closed position, where the earplug is positioned proximal to the ear of the user to at least partly block the ambient sound and allow the user to hear the ambient sound at a reduced volume that is less than the unchanged volume. A reduced volume as used herein comprises a noise reduction rating (NRR) of at least about 10 decibels to at most about 35 decibels. In the example where the hearing protection system includes a trigger mechanism employing a sensor to continually sense the position of a firearm relative to a shoulder of the hunter, the trigger mechanism can cause the earplug to be displaced between the open position and the closed position based at least partly on a position of the end or butt of the stock of a long gun (e.g., shotgun, rifle, muskets, etc.) relative to the shoulder of the hunter. For example, the trigger mechanism can employ a sensor (a touch sensor, an electro-optical sensor, a magnetic proximity sensor, radio-frequency identification (RFID), etc.) arranged with the shoulder of the hunter. The sensor to detect the butt of the stock of the long gun in response to a proximity of the butt of the long gun to the sensor arranged with the shoulder. In the example where the hearing protection system includes a trigger mechanism employing a sensor to continually sense the position of a firearm relative to a hand of the hunter, the trigger mechanism can cause the earplug to be displaced between the open position and the closed position based at least in part on a position of a grip and/or a trigger of a handgun (e.g., a pistol, a revolver, a pistol revolver, a revolving pistol, etc.) relative to a finger and/or a palm of the hand of the hunter. The sensor to detect the grip and/or the trigger of the handgun in response to a proximity of the grip and/or trigger of the handgun to a sensor arranged with the finger and/or the palm of the hand of the hunter.

Further, the hearing protection system may include an earplug attachable proximate to an ear of a user. For example, the hearing protection system may include a receptacle receivable by the ear of the user, and the earplug may be displaceably attached to the receptacle. The earplug may be displaceably attached to the receptacle such that when in the closed position the earplug is removeably received by a cavity of the receptacle to at least partly seal the cavity of the receptacle and to at least partly block ambient sound and allow the user to hear the ambient sound at a reduced volume.

The hearing protection systems may include a motor coupled to the earplug. The motor being coupled to the earplug to displace the earplug between the open positon and the closed position based at least in part on a signal received from the trigger mechanism. For example, the motor may receive a wired or a wireless signal from the trigger mechanism that causes the motor to displace the earplug between the open position and the closed potion.

### Illustrative Hearing Protection Systems

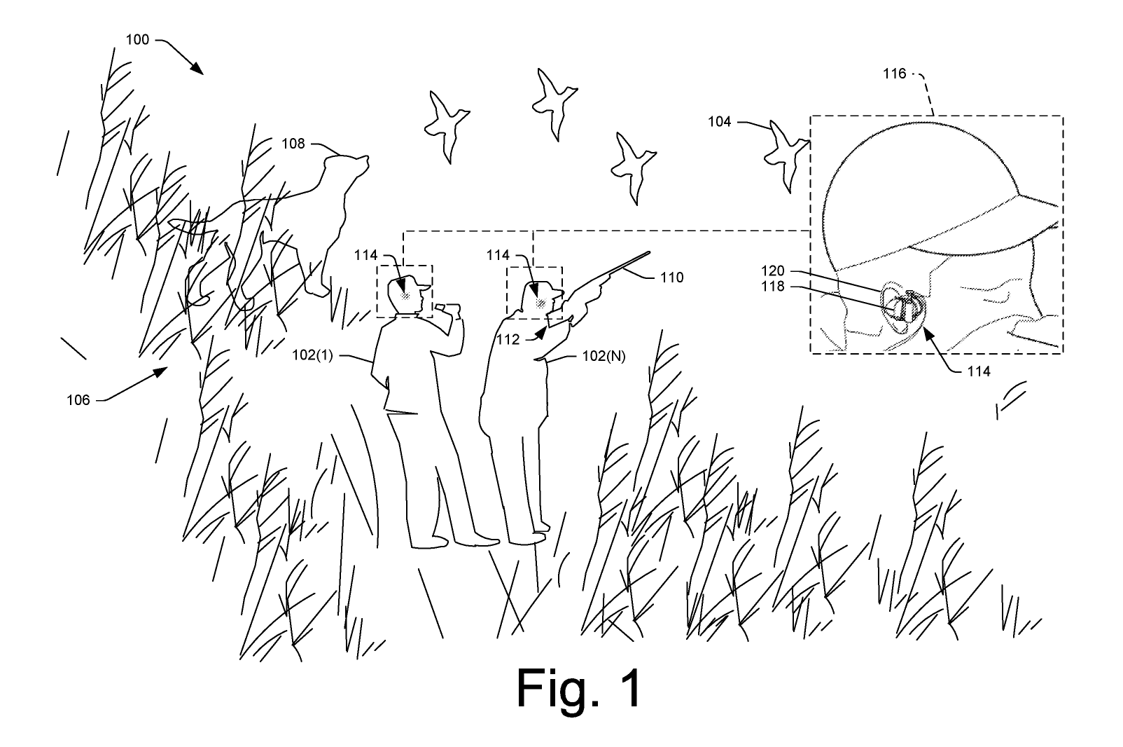

FIG. 1 illustrates an example hunting environment 100 involving hunters 102(1) and 102(N). For example, FIG. 1 illustrates a hunting environment 100 of the hunters 102(1) and 102(N) attempting to shoot game 104 flushed out of tall grass 106 by a dog 108. Further, while attempting to shoot the game 104 with a firearm 110, a butt 112 of the firearm 110 is “braced” or forced onto the shoulder of the hunter 102(N) to fire the firearm 110 at the game 104. While FIG. 1 illustrates a hunting environment 100 where the butt 112 of the firearm 110 is braced into the shoulder of the hunter, other types of hunting environments may occur. For example, a hunting environment may occur where a grip of a handgun is grasped by a palm of a hand of the hunter, or trigger of a handgun is touched by a finger of a hand of the hunter. In most cases, the firearm 110 is positioned to fire at the game 104 relative to a portion of the body of the hunter. As a result of the firearm 110 being positioned to fire at the game 104, a hearing protection system 114 used by the hunters 102(1) and 102(N) are displaced from an open position to a closed position to protect the ears of the hunters 102(1) and 102(N) from the noise produced from firing the firearm 110.

Detail view 116 illustrates the hearing protection system 114 in more detail, and shows an earplug 118 of the hearing protection system 114 arranged with the ears 120 of the hunters 102(1) and 102(N). Detail view 116 illustrates the earplug 118 of the hearing protection system 114 arranged to protect the hearing of the hunters 102(1) and 102(N) from the noise produced from firing the firearm 110 at the game 104. For example, detail view 116 illustrates the earplug 118 of the hearing protection system 114 displaced from the open position to the closed position as a result of the firearm 110 being positioned to fire at the game 104.

When the hearing protection system 114 is in the open position, the earplugs 118 are positioned away from the ears 120 of the hunters 102(1) and 102(N) to allow the hunters 102(1) and 102(N) to hear ambient sound at an unchanged volume. For example, when in the open position, the earplugs 118 are positioned away from the ears 120 of the hunters 102(1) and 102(N) to allow the hunters 102(1) and 102(N) to hear, naturally in the hunting environment 100, the dog 108 moving in the tall grass 106, the game 104 being flushed out of the tall grass 106, and verbal communication between the hunters 102(1) and 102(N) until the firearm 110 is moved into position to fire at the game 104. Because the earplugs 118 are positioned away from the ears 120 of the hunters 102(1) and 102(N), until the firearm 110 is positioned to be fired, the hearing protection system 114 allows the hunters 102(1) and 102(N) to hear the ambient sound naturally at an unchanged volume making the hearing protection system 114 more efficient and safe to use than earplugs that block the ambient sound, and more efficient and safe to use than noise cancelling earplugs that inject reproduced ambient sound into the ears. Moreover, because the earplugs 118 are positioned away from the ears 120 of the hunters 102(1) and 102(N) until the firearm 110 is positioned to fire at the game 104, the earplugs 118 allow heat to dissipate from the ears 120, and thus do not cause the ears 120 to perspire or the hunters 102(1) and 102(N) to overheat, making the hearing protection system 114 more comfortable to use than the earplugs and the noise cancelling earplugs that remain stuck in the ears of a user even when the earplugs and the noise cancelling earplugs are not needed.

When the firearm 110 is positioned to fire at the game 104, the hearing protection system 114 is in the closed position and the earplugs 118 are positioned proximal to the ears 120 of the hunters 102(1) and 102(N) to at least partly block the ambient sound and allow for the hunters 102(1) and 102(N) to hear the ambient sound at a reduced volume (e.g., a noise reduction of at least about 10 decibels to at most about 35 decibels) that is less than the unchanged volume. For example, when the firearm 110 is positioned to fire at the game 104, the earplugs 118 can be positioned proximal to the ears 120 of the hunters 102(1) and 102(N) to reduce the firing noise of the firearm received by the ears 120 of the hunters 102(1) and 102(N) by at least about 10 decibels to at most about 35 decibels to protect the hearing of the ears 120 of the hunters 102(1) and 102(N).

While FIG. 1 illustrates the hearing protection systems 114 arranged in the ears 120 of the hunters 102(1) and 102(N), the hearing protection systems 114 may not be arranged in the ears 120. For example, the hearing protection system 114 may be arranged with a headband, a muff, a helmet, a hat, a cap, a stocking cap, eyewear, etc. arranged to fit on the head of the user and interface with the ears of the user. Moreover, while FIG. 1 illustrates the hearing protection systems 114 including earplugs 118 arranged to fit in the ears 120 of the hunters 102(1) and 102(N), the hearing protection system 114 may include ear muffs, ear cups, ear pads, etc. arranged to fit on and/or over the ears 120 of the user.

The hearing protection system 114 may use a trigger mechanism, responsive to movement of the firearm 110, to cause the earplugs to displace between the open position and the closed position based at least in part on a position of the firearm 110. For example, the trigger mechanism may include a sensor arranged with a portion of the body of the hunter, and the trigger mechanism, responsive to movement of the firearm 110, causes the earplug 118 to displace between the open position and the closed position based at least in part on the position of the firearm 110 relative to the portion of the body of the hunter. For example, a sensor can be arranged with a shoulder of the hunter 102(N) that detects the butt 112 of the firearm 110 in response to the butt 112 of the firearm 110 being at a position relative the shoulder of the hunter 102(N). If the position of the butt 112 of the firearm 110 is positioned close enough to the shoulder of the hunter 102(N), the trigger mechanism may displace the earplugs 118 of the hunters 102(1) and 102(N). For example, the sensor may be any device that measures the position of the butt 112 of the firearm 110 relative to the shoulder of the hunter 102(N), and converts that position to a wired or a wireless signal that controls a motor that displaces the earplugs 118 from the open position to the closed position. The sensor may be a touch sensor, an electro-optical sensor, a magnetic proximity sensor, etc. For example, a touch sensor, an electro-optical sensor, a magnetic proximity sensor, etc. may be arranged with the shoulder of the hunter 102(N) and/or the butt 112 of the firearm 110 that senses when the butt 112 of the firearm 110 is within a threshold distance to the shoulder of the hunter 102(N).

FIG. 1 illustrates the hearing protection systems 114 can be connected to one or more hunters 102(1) and 102(N). For example, the hearing protection systems 114 of the hunters 102(1) and 102(N) may be communicatively coupled via wireless interconnection. For example, the wireless interconnection interconnecting the hearing protection systems 114 of the hunters 102(1) and 102(N) may be a radio interconnection, an infrared interconnection, a Bluetooth interconnection, etc. In an example where the hearing protection system 114 of the hunter 102(N) is wirelessly interconnected to the hearing protection system 114 of the hunter 102(1), the hearing protection system 114 of the hunter 102(N) can signal the hearing protection system 114 of the hunter 102(1) to displace from the open position to the closed position to protect the hearing of the hunter 102(1) from the noise produced from the hunter 102(N) firing the firearm 110. For example, the hearing protection systems 114 can include a transmitter, transceiver, antenna, etc. arranged with the trigger mechanisms of the hearing protection systems 114. The trigger mechanism of the hearing protection system 114 of the hunter 102(N) can send a signal to the hearing protection system 114 of the hunter 102(1). When the trigger mechanism of the hearing protection system 114 of the hunter 102(N) responds to movement of the firearm 110, and causes the earplugs 118 of the hunter 102(N) to displace between the open position and the closed position based at least in part on the position of the firearm 110 relative to the portion of the body of the hunter 102(N), the hearing protection system 114 of the hunter 102(N) can send a signal to the trigger mechanism of the hearing protection system 114 of the hunter 102(1) to displace the earplugs 118 of the hunter 102(1) between the open position and the closed position.

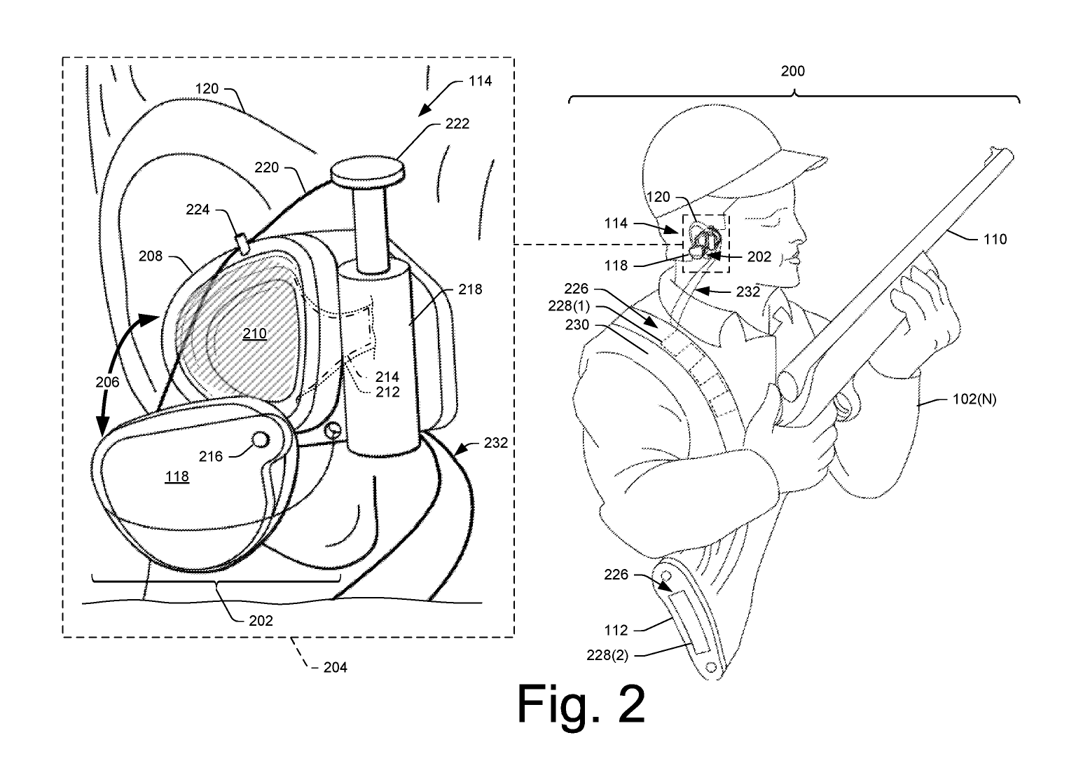

FIG. 2 illustrates a perspective view 200 of the example hearing protection system 114 shown in FIG. 1 in an open position 202 that allows the hunters 102(1) and 102(N) shown in FIG. 1 to hear ambient sound at an unchanged volume.

Detail view 204 illustrates the hearing protection system 114 in the open position 202 in more detail, and shows the earplug 118 positioned a distance 206 away from the ear 120 of the hunter 102(N) to allow the hunter 102(N) to hear ambient sound at an unchanged volume. The distance 206 the earplug 118 can be positioned away from the ear 120 may be at least about 0.06 inches (0.2 centimeters) to at most about 5 inches (13 centimeters).

Detail view 204 shows the hearing protection system 114 can include a receptacle 208. The receptacle 208 can be receivable by the ear 120. For example, the receptacle 208 can be a hollow earplug or hollow earpiece that can be received by the ear 120 of a user. The receptacle 208 can have a wall thickness of at least about 0.005 inches (0.01 centimeters) to at most about 0.02 inches (0.05 centimeters). The receptacle 208 can be formed of plastic, rubber, metal, wood, carbon fiber, fiberglass, glass, composite, etc. The receptacle 208 can include a cavity 210 arranged to fit in at least a portion of the ear 120. For example, the cavity 210 may be arranged in the receptacle 208 such that the cavity 210 interfaces with the inside the ear 120 proximate to the opening of the canal 212 (e.g., auditory canal) (illustrated as a hidden dashed line) of the ear 120. The receptacle 208 can include a channel 214 (illustrated as a hidden dashed and dotted line) communicatively coupled to the cavity 210 and arranged to fit in at least a portion of the canal 212 of the ear 120. The channel 214 may be arranged to extend a distance of at least about 0.1 inches (0.02 centimeters) to at most about 0.5 inches (1 centimeter) into the canal 212 of the ear 120.

Detail view 204 shows the earplug 118 is displaceably attached, via a hinge 216, to the receptacle 208. While detail view 204 shows the hinge 216 is rotatably attaching the earplug 118 to the receptacle 208, the hinge 216 may foldably, pivotably, slideably, etc. attach the earplug 118 to the receptacle 208. While detail view 204 shows the earplug 118 displaceably attached, via the hinge 216, to the receptacle 208, the earplug 118 may not be displaceably attached via the hinge 216. For example, the earplug 118 may be displaceably attached to the receptacle 208 via a hydraulic actuator, pneumatic actuator, magnetic actuator, etc. Further, while FIG. 2 illustrates the earplug 118 displaceably attached to the receptacle 208, the earplug 118 may not be displaceably attached to the receptacle 208. For example, the earplug 118 may be displaceably attached to a headband, a muff, a helmet, a hat, a cap, a stocking cap, eyewear, etc. arranged to fit on the head of the user.

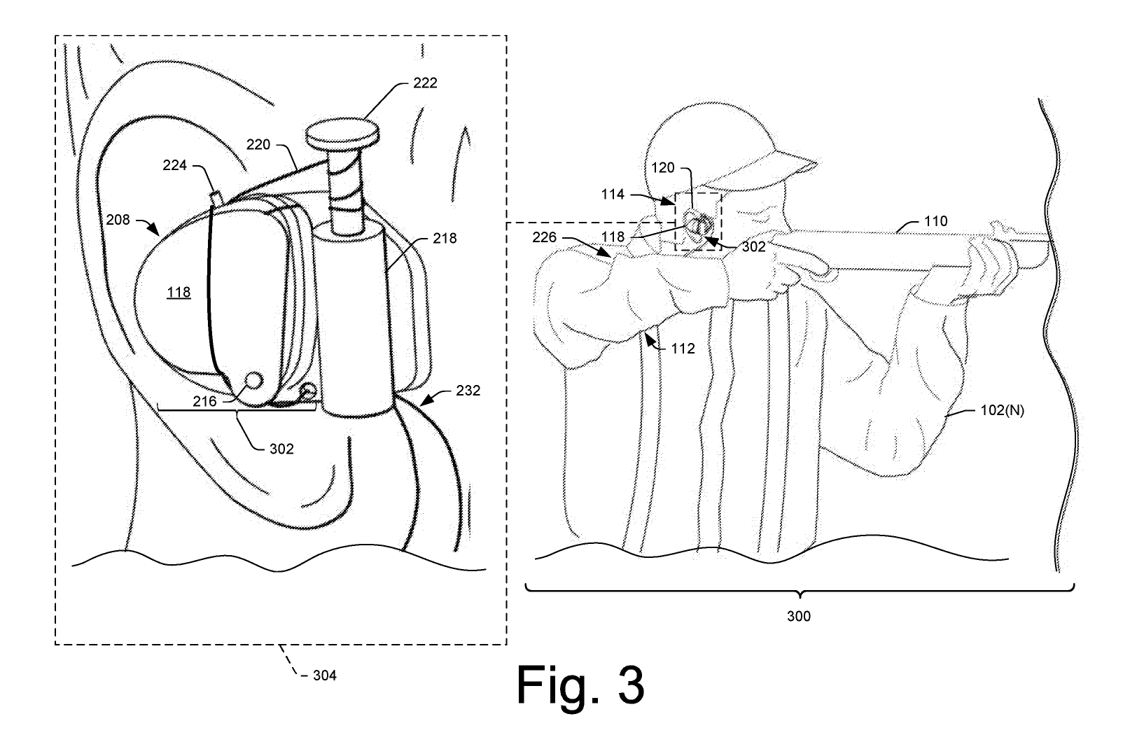

Detail view 204 shows a motor 218 coupled to the earplug 118. For example, the motor 218 can be fixed to the receptacle 208 and coupled, via a member 220, to the earplug 118. The member 220 can be a line, wire, a chain, etc. having first end fixed to the earplug 118 and second end fixed to a rotating shaft 222 of the motor 218. When the motor 218 rotates the rotating shaft 222, the member 220 wraps around the rotating shaft 222. As the member 220 wraps around the rotating shaft 222, the member 220 displaces the earplug 118 from the open positon 202 to the closed position (described in detail with regard to FIG. 3 below). Detail view 204 shows a guide member 224 fixed to the receptacle 208 and arranged to guide the member 220 as the member 220 is displaced via the rotating shaft 222. The guide member 224 can be a post, a hook, a ring, a ferrule, etc. arranged to guide the member 220 as the member 220 is displaced via the rotating shaft 222.

FIG. 2 illustrates a trigger mechanism 226 can have a first sensor 228(1) arranged with a shoulder 230 of the hunter 102(N) and a second sensor 228(2) arranged with the butt 112 of the firearm 110. The first and second sensors 228(1) and 228(2) measure the position of the butt 112 of the firearm 110 relative to the shoulder 230 of the hunter 102(N), and can convert that position to a wired signal 232. The wired signal 232 controls the motor 218 that displaces the earplugs 118 from the open position 202 to the closed position. While FIG. 2 illustrates the trigger mechanism 226 having first and second sensors 228(1) and 228(2) that convert the position of the butt 112 of the firearm 110 relative to the shoulder 230 of the hunter 102(N) to a wired signal 232, the first and second sensors 228(1) and 228(2) may convert the position to a wireless signal. For example, the first sensor 228(1) and/or the second sensor 228(2) may convert the position to a radio signal, an infrared signal, a Bluetooth signal, etc. that controls the motor 218 that displaces the earplugs 118 from the open position 202 to the closed position. The first and second sensors 228(1) and 228(2) may be touch sensors, electro-optical sensors, magnetic proximity sensors, etc. In an example where the first sensor 228(1) and/or the second sensor 228(2) may convert the position to a radio signal and/or a Bluetooth signal, the firearm 110 can have an accelerometer that could determine whether the firearm 110 was in a non-use position (e.g., vertical in rest position) or in a use position (e.g., horizontal in fire position). In another example where the first sensor 228(1) and/or the second sensor 228(2) may convert the position to a radio signal and/or a Bluetooth signal, the firearm 110 can have an accelerometer that senses a rapid change from a first position to a second position. Further, in another example where the first sensor 228(1) and/or the second sensor 228(2) may convert the position to a radio signal and/or a Bluetooth signal, the firearm 110 can have an accelerometer that communicates the position to the hearing protection system 114, via a device (e.g., a small computing device, a mobile device, a handheld computer, a handheld, etc.). For example, an accelerometer arranged with the firearm 110 may communicate the position of the firearm 110 to a smartphone of the user, and the smartphone may communicate the communicated position to the hearing protection system 114.

FIG. 3 illustrates a perspective view 300 of the example hearing protection system 114 shown in FIG. 2 in a closed position 302 that allows the hunters 102(1) and 102(N) shown in FIG. 1 to hear ambient sound at a reduced volume that is less than the unchanged volume.

Detail view 304 illustrates the hearing protection system 114 in the closed positon 302 in more detail, and shows the earplug 118 is positioned proximal to the ear 120 of the hunter 102(N) to at least partly block the ambient sound and allow the hunter 102(N) to hear the ambient sound at a reduced volume that is less than the unchanged volume. For example, the earplug 118 can be positioned in the ear 120 to at least partly block the ambient sound and allow the hunter 102(N) to hear the ambient sound at a reduced volume that is less than the unchanged volume. For example, when the earplug 118 is in the closed position 302, the earplug 118 can be removeably received by the cavity 210 of the receptacle 208 to at least partly seal the cavity 210 of the receptacle 208 and at least partly block the ambient sound and allow the hunter 102(N) to hear the ambient sound at the reduced volume. In another example, when the earplug 118 is in the closed position 302, the earplug 118 can be removeably received by the cavity 210 of the receptacle 208 to at least partly seal the channel 214 of the receptacle 208 and at least partly block the ambient sound and allow the user to hear the ambient sound at the reduced volume. The earplug 118 can be a cap, a flap, a plug, etc. arranged to cover the receptacle 208, or the cavity 210 of the receptacle 208. Moreover, the earplug 118 can be arranged to fit on or over the ear 120. For example, the earplug 118 can be an ear muff, an ear cup, an ear pad, etc. arranged to fit on or over the ear 120.

FIG. 3 illustrates the trigger mechanism 226 having caused the earplug 118 to displace from the open position 202 to the closed position 302 based at least in part on the position of the firearm 110 relative to the shoulder 112 the hunter 102(N). For example, FIG. 3 illustrates the first and second sensors 228(1) and 228(2) having measured the position of the butt 112 of the firearm 110 relative to the shoulder 230 of the hunter 102(N), and having converted that position to the wired signal 232. The wired signal 232 having controlled the motor 218 that displaced the earplug 118 from the open position 202 to the closed position 302. While FIG. 3 illustrates the first and second sensors 228(1) and 228(2) converting the position to the wired signal 232, the first and second sensors 228(1) and 228(2) may convert the position to a wireless signal that controls the motor 218 that displaces the earplug 118 from the open position 202 to the closed position 302.

### Conclusion

Although the invention has been described in language specific to structural features and/or methodological acts, it is to be understood that the invention is not necessarily limited to the specific features or acts described. Rather, the specific features and acts are disclosed as illustrative forms of implementing the invention. For example, while embodiments are described having certain shapes, sizes, and configurations, these shapes, sizes, and configurations are merely illustrative.

1. A hearing protection system comprising:
an earplug displaceable between an open position and a closed position;
a receptacle receivable by an ear of a user, the receptacle comprising a cavity arranged to fit in at least a portion of the ear, wherein:
when in the open position, the earplug is positioned away from the ear of the user to allow the user to hear ambient sound at an unchanged volume, and
when in the closed position, the earplug is removeably received by the cavity of the receptacle and at least partly seals the cavity of the receptacle to at least partly block the ambient sound and allow the user to hear the ambient sound at a reduced volume that is less than the unchanged volume; and
a trigger mechanism, responsive to movement of a device, to cause the earplug to displace between the open position and the closed position based at least in part on a position of the device.

2. The hearing protection system of claim 1, wherein the device comprises a firearm, the trigger mechanism comprises a sensor arranged with a portion of the body of the user, and the trigger mechanism causes the earplug to displace between the open position and the closed position based at least in part on the position of the firearm relative to the portion of the body of the user.

3. The hearing protection system of claim 2, wherein the firearm comprises a long gun, the portion of the body comprises a shoulder of the user, and the trigger mechanism causes the earplug to displace between the open position and the closed position based at least in part on the position of the long gun relative to the shoulder of the user.

4. The hearing protection system of claim 2, wherein the firearm comprises an handgun, the portion of the body comprises a portion of at least one hand of the user, and the trigger mechanism causes the earplug to displace between the open position and the closed position based at least in part on the position of the handgun relative to the portion of the at least one hand of the user.

5. (canceled)

6. The hearing protection system of claim 1, further comprising a motor coupled to the earplug, wherein the motor displaces the earplug between the open positon and the closed position based at least in part on a wired or wireless signal received from the trigger mechanism.

7. A hearing protection system comprising:
a receptacle receivable by an ear of a user, the receptacle comprising a cavity arranged to fit in at least a portion of the ear;
an earplug displaceably attached to the receptacle attachable proximate to an ear of a user; and
a trigger mechanism arrangeable with a portion of the body of the user, the trigger mechanism, responsive to movement of a device, causing the earplug to displace between an open position and a closed position based at least in part on a position of the device, wherein
when in the open position, the earplug is positioned away from the ear of the user to allow the user to hear ambient sound at an unchanged volume, and
when in the closed position, the earplug is removeably received by the cavity of the receptacle to at least partly seal the cavity of the receptacle positioned proximal to the car of the user to at least partly block the ambient sound and allow the user to hear the ambient sound at a reduced volume that is less than the unchanged volume.

8. (canceled)

9. The hearing protection system of claim 7, wherein the receptacle further comprises a channel communicatively coupled to the cavity and arranged to fit in at least a portion of an auditory canal of the ear, and
wherein the earplug is displaceably attached to the receptacle such that when in the closed position, the earplug is removeably received by the cavity of the receptacle to at least partly seal the channel of the receptacle and to at least partly block the ambient sound and allow the user to hear the ambient sound at the reduced volume.

10. The hearing protection system of claim 7, further comprising a motor coupled to the earplug, wherein the motor displaces the earplug between the open positon and the closed position based at least in part on a wired or wireless signal received from the trigger mechanism.

11. The hearing protection system of claim 7, wherein the device comprises a firearm, the trigger mechanism comprises a sensor arranged with a portion of the body of the user, and the trigger mechanism, responsive to movement of the device, causes the earplug to displace between the open position and the closed position based at least in part on the position of the firearm relative to the portion of the body of the user.

12. The hearing protection system of claim 7, wherein the trigger mechanism comprises a touch sensor, an electro-optical sensor, or a magnetic proximity sensor.

13. A hearing protection system comprising:
a receptacle including a cavity arranged to fit in at least a portion of an ear of a user;
an earplug displaceably attached to the receptacle, the earplug displaceable from an open position to a closed position in response to a signal received from a trigger mechanism,
wherein when in the open position, the earplug is positioned away from an ear of a user and to allow the user to hear ambient sound at an unchanged volume, and when in the closed position, the earplug is removeably received by the cavity of the receptacle and at least partly seals the cavity of the receptacle positioned proximal to the car of the user to at least partly block the ambient sound and allow the user to hear the ambient sound at a reduced volume that is less than the unchanged volume.

14. The hearing protection system of claim 13, wherein the trigger mechanism comprises a sensor that causes the earplug to displace from the open positon to the closed position based at least in part on a position of a device.

15. The hearing protection system of claim 14, wherein the user is a first user, and wherein the sensor is arranged with a portion of a body of a second user to cause the earplug to displace from the open position away from the ear of the first user to the closed position on the ear of the first user based at least in part on the position of the device relative to the portion of the body of the second user.

16. The hearing protection system of claim 14, wherein the sensor is arranged with a portion of the body of the user to cause the earplug to displace from the open position to the closed position based at least in part on the position of the device relative to the portion of the body of the user.

17. The hearing protection system of claim 14, wherein the device comprises a firearm.

18. (canceled)

19. The hearing protection system of claim 13, further comprising a motor coupled to the earplug, wherein the motor displaces the earplug between the open positon and the closed position based at least in part on a wired or wireless signal received from the trigger mechanism.

20. The hearing protection system of claim 13, wherein the trigger mechanism comprises a touch sensor, an electro-optical sensor, or a magnetic proximity sensor.
